(19) United States
(12) Patent Application Publication
(10) Pub. No.: US 2007/0105924 A1
(43) Pub. Date: May 10, 2007

(54) Vigabatrin bioisoteres and related methods of use

(76) Inventors: Richard B. Silverman, Northbrook, IL (US); Hai Yuan, Chicago, IL (US)

Correspondence Address: REINHART BOERNER VAN DEUREN S.C.;ATTN: LINDA KASULKE, DOCKET COORDINATOR, 1000 NORTH WATER STREET, SUITE 2100, MILWAUKEE, WI, 53202, US

(21) Appl. No.: 11/526,307

(22) Filed: Sep. 25, 2006

Related U.S. Application Data
(60) Provisional application No. 60/719,868, filed on Sep. 23, 2005.

(51) Int. Cl. A61K 31/41; C07D 257/02

(52) U.S. Cl. 514/381; 548/253

(57) ABSTRACT

Compounds bioisoteric to vigabatrin and related methods of use.

This invention claims priority benefit from application Ser. No. 60/719,868 filed Sep. 23, 2005, the entirety of which is incorporated herein by reference.

The United States Government has certain rights to this invention pursuant to Grant No. GM66132 from the National Institutes of Health to Northwestern University.

### BACKGROUND OF THE INVENTION

γ-Aminobutyric acid aminotransferase (GABA-AT) is a pyridoxal-5′-phosphate (PLP)-dependent enzyme that degrades the major inhibitory neurotransmitter γ-aminobutyric acid (GABA) in the central nervous system (CNS, Scheme 1). GABA is important to several neurological disorders, including Parkinson's disease, Huntington's chorea, Alzheimer's disease, and epilepsy, a central nervous system disease characterized by recurring convulsive seizures. A deficiency of GABA in the brain has been implicated as one cause for convulsions. (Karlsson, A.; Funnum, F.; Malthe-Sorrensen, D.; Storm-Mathisen, J. Biochem Pharmacol 1974, 22, 3053-3061.) In an effort to raise the concentration of GABA in the brain, both direct injection and oral administration of GABA have been studied. It was shown that injection of GABA into the brain has an anticonvulsant effect, but it is obviously not a practical method. Taking GABA orally, however, is not effective because GABA cannot cross the blood-brain barrier, a membrane protecting the CNS from xenobiotics in the blood.

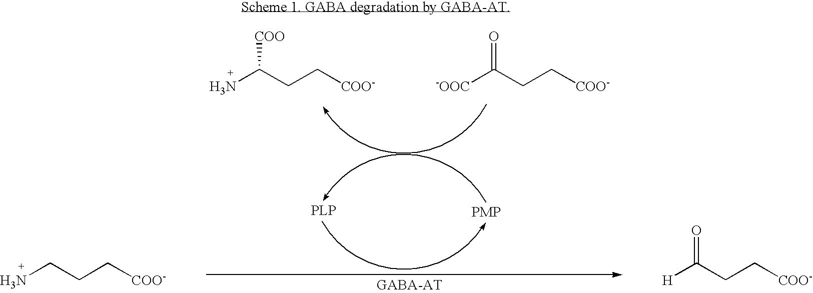

To correct the deficiency of brain GABA and therefore stop convulsions, an important approach is to use an inhibitor of GABA-AT that is able to cross blood-brain barrier. (Nanavati, S. M.; Silverman, R. B. J. Med. Chem. 1989, 32, 2413-2421.) Inhibition of this enzyme increases the concentration of GABA in the brain and could have therapeutic applications in epilepsy as well as other neurological disorders. One of the most effective in vivo time-dependent inhibitors of GABA-AT is 4-amino-5-hexenoic acid (FIG. 1, vigabatrin, 1), an anticonvulsant drug marketed all over the world except in the U.S.

Various analogues of vigabatrin as inhibitors of GABA-AT have been prepared, but all such compounds contain the same hydrophilic carboxylic acid group found in vigabatrin. Inasmuch as lipophilicity is an important factor influencing the ability of a compound to permeate the blood-brain barrier, the art continues the search for an effective, potent vigabatrin analogue with improved lipophilicity.

### SUMMARY OF THE INVENTION

In light of the foregoing, it is an object of the present invention to provide various compounds and/or compositions and related methodologies for the inactivation and/or inhibition of γ-aminobutyric acid aminotransferase, such inactivation or inhibitory activity as can be used in the treatment of convulsions, epilepsy and other CNS disease states, such as neurodegenerative diseases, thereby overcoming various deficiencies and shortcomings of the prior art, including those outlined above. It will be understood by those skilled in the art that one or more aspects of this invention can meet certain objectives, while one or more other aspects can meet certain other objectives. Each objective may not apply equally, in all its respects, to every aspect of this invention. As such, the following objects can be viewed in the alternative with respect to any one aspect of this invention.

It is an object of the present invention to provide a mechanism-based inhibition and/or inactivation methodology and one or more compounds useful in conjunction therewith.

It is another object of the present invention to provide one or more compounds demonstrating inhibitory activity with respect to γ-aminobutyric acid aminotransferase, such activity as would be understood by those skilled in the art to be efficacious in the treatment of epilepsy and other disease states characterized by convulsive seizures.

It is another object of the present invention to provide one or more compounds incorporating rationally-designed structural characteristics consistent with mechanism-based inhibition/inactivation of γ-aminobutyric acid aminotransferase, such compounds having incorporated therein a moiety to enhance lipophilicity, as compared to the prior art, to facilitate transport.

Other objects, features, benefits and advantages of the present invention will be apparent from this summary and its descriptions of certain embodiments, and will be readily apparent to those skilled in the art having knowledge of various enzyme systems, and inhibitory compounds and their preparation. Such objects, features, benefits and advantages will be apparent from the above as taken into conjunction with the accompanying examples, data, figures and all reasonable inferences to be drawn therefrom.

In part, the present invention can comprise a γ-aminobutyric acid aminotransferase inhibitor compound of a formula 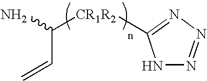
wherein n can be an integer ranging from 1 to about 6. R₁ and R₂ can be independently selected from H, alkyl and substituted alkyl moieties. Alternatively, such inhibitors can be tautomers and/or salts of such a compound; that is, including but not limited to an ammonium salt of such a compound. Regardless, with regard to stereochemistry, any such compound can have either an R or S configuration.

As shown below in the context of several synthetic preparations, in certain embodiments of the present inhibitor compounds, n can be 1, 2 or 3 and any such compound can be provided as a salt. In certain such embodiments, the counter ion can be the conjugate base of a protic acid. Without limitation, certain embodiments of this invention can comprise the ammonium hydrochloride salt of any such compound. Regardless of n, stereochemistry, salt or tautomer, in certain embodiments R₁ and R₂ can be H.

As discussed more fully below in conjunction with known and accepted mechanistic considerations, the present invention can also include a complex comprising the addition product of a γ-aminobutyric acid aminotransferase and a compound of this invention, such a complex inactivating or inhibiting the enzyme component thereof. Without limitation, such compounds can include those discussed more fully above and illustrated below, all as can be varied in accordance within the range of stereochemical relationships contemplated within the broader aspects of this invention. As would be understood by those skilled in the art, the enzyme component of such an addition product can further comprise a pyridoxal-5′-phosphate cofactor.

Accordingly, the present invention can also include a method of inhibiting a γ-aminobutyric acid aminotransferase. Such a method can comprise contacting the enzyme with at least a partially effective amount of one of the aforementioned compounds. Such contact can be, as would be understood by those skilled in the art, experimentally and/or for research purposes or as may be designed to simulate one or more in vivo or physiological conditions. In certain embodiments, inhibition can be achieved with one or more compounds where n can range from 1 to about 6. In certain other embodiments, n can be 1, 2 or 3, and R₁ and R₂ can be H. Regardless, the amino and tetrazole moieties can vary by degree of protonation and the presence of a corresponding salt. Likewise, such compounds are considered without limitation as to stereochemistry.

Moreover, in yet another departure from the prior art, the present invention can provide a method of using a tetrazole moiety to enhance the lipophilicity of a γ-aminobutyric acid aminotransferase inhibitor. Such a method can comprise providing a compound from a group of compounds of a formula 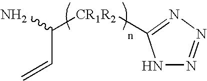
wherein n can range from 1 to about 6; such compounds including tautomers and salts thereof; and determining the lipophilicity of such a compound as compared to vigabatrin. Such compounds can be of the sort described above and illustrated elsewhere, herein, and can vary within the full range of possible structural, ionic and/or stereochemical considerations.

### BRIEF DESCRIPTION OF THE DRAWINGS

FIG. 1 shows the structure of vigabatrin (prior art).

FIG. 2 shows structures of several vigabatrin bioisoteres, in accordance with certain non-limiting embodiments of this invention.

FIG. 3 shows structures of another bioisotere and alkyl derivatives thereof, in accordance with certain non-limiting embodiments of this invention.

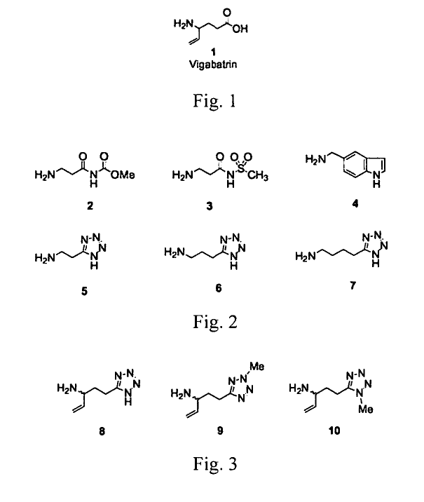

### DETAILED DESCRIPTION OF CERTAIN EMBODIMENTS

En route to representative compounds and methods of this invention, a series of potential substrates of GABA-AT was designed by replacing the carboxylic acid group with more lipophilic bioisosteres (FIG. 2, compounds 2-5). In order to fit the potential substrates containing bioisosteric groups larger than the carboxylic acid group of GABA into the small and constricted active site of the enzyme, P-alanine, another natural substrate of GABA-AT containing one less methylene group than GABA, was selected as the parent structure.

Compound 2 was selected because it contains an isosteric functionality that is less acidic (pKₐ˜8) than that of a carboxylic group; compound 3 has a pKₐ value comparable to that of a carboxylic acid. Compound 4 contains an indole ring, which may be able to participate in a π-cation interaction with Arg-192, the residue to which the carboxylic acid group of GABA binds. Compound 5 was also considered because of the biological compatibility of its tetrazole group. To optimize the carbon chain length, tetrazole derivatives 6 and 7 with one and two additional methylenes, respectively, were also made.

Based on the structure of 6, compound 8, a tetrazole bioisostere of the antiepilepsy drug vigabatrin, was synthesized. N-Methyl tetrazole derivatives 9 and 10 were also made and tested to determine the form of the tetrazole ring in the active site of the enzyme (FIG. 3). Herein we report the syntheses and the enzymatic results with these compounds.

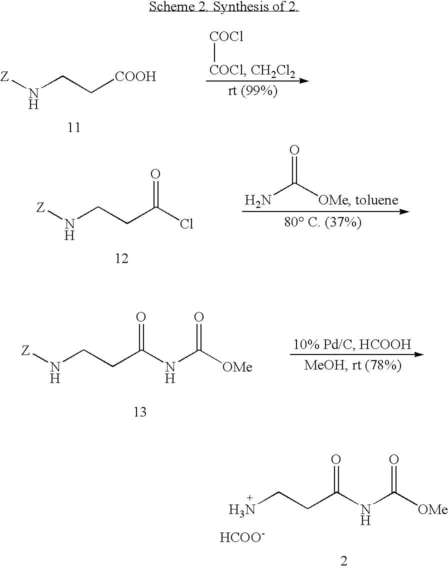

Methyl β-alanylcarbamate (2) was made from N-Cbz-β-alanine (11) as shown in Scheme 2. Compound 11 was treated with oxalyl chloride to give acyl chloride 12, which was allowed to react with methyl carbamate to give methyl N-Cbz-β-alanylcarbamate (13). Catalytic transfer hydrogenation using formic acid and 10% palladium on active carbon gave 2 in the form of a formate salt.

Methyl β-alanylsulfonamide (3) was synthesized as shown in Scheme 3. Protected β-alanine 11 was treated with carbonyldiimidazole to give 14, which was allowed to react with methanesulfonamide in the presence of 1,8-diazabicyclo[5.4.0]undec-7-ene (DBU) to afford 15. Deprotection of the Cbz group with 30% HBr in acetic acid provided the desired 3 in the form of a hydrobromide salt.

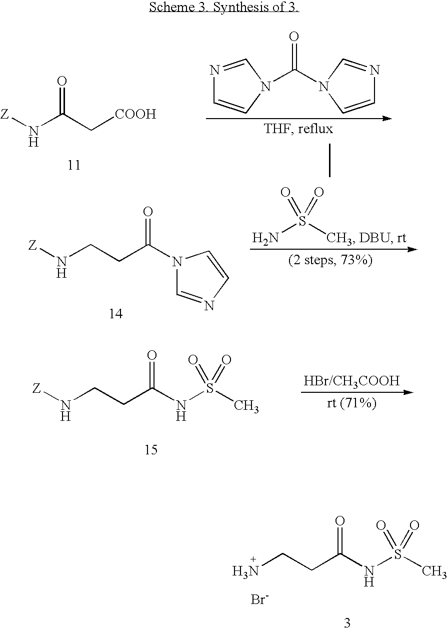

Indole-5-methanamine (4) was prepared from 5-cyanoindole (16) by reduction with LiAlH₄ (Scheme 4).

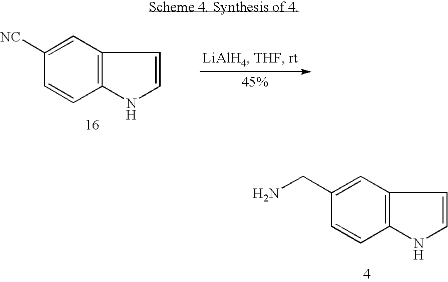

The synthesis of 1H-tetrazole-5-ethanamine (5) is shown in Scheme 5. 3-Aminopropionitrile (17) was treated with benzyl chloroformate and sodium hydroxide to give N-Cbz-3-aminopropionitrile (18). The reaction of 18 with sodium azide in the presence of triethylammonium hydrochloride afforded N-Cbz-aminoethyltetrazole (19). Deprotection provided desired compound 5.

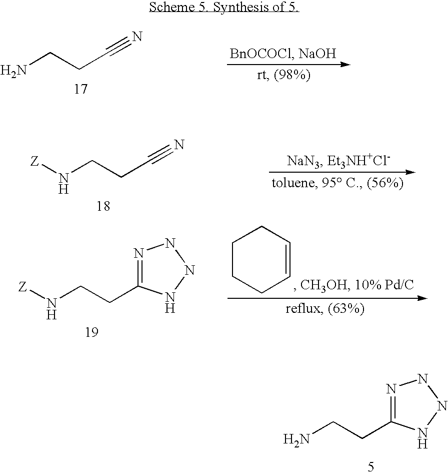

Preliminary substrate activity tests using [⁴C]-labeled α-ketoglutarate (α-KG) showed that compounds 2, 3, and 5 are substrates for GABA-AT; 4, however, showed an α-KG conversion of only 0.1%, indicating that 4 has little or no substrate activity (Table 1). Tetrazole derivative 5 was the most efficient of the synthesized substrates. The tetrazole group was the best bioisostere for the carboxylic acid group in this series of compounds tested.

TABLE 1
Preliminary substrate activity test results

| | Compound | | | | |
|---|---|---|---|---|---|
| | GABA | 2 | 3 | 4 | 5 |
| Converted α-KG^{a} | 25% | 1.3% | 6.4% | 0.10% | 20% |

^{a}incubation time 48 h, GABA-AT: 0.7 μM, substrate: 2.5 mM, α-KG: 2.9 mM.

To determine the optimal carbon chain length of the tetrazole derivatives, compounds 6 and 7 with one and two additional methylene groups, respectively, than 5 were synthesized. 1H-Tetrazole-5-propanamine (6) was synthesized from 4-bromobutyronitrile (20) as shown in Scheme 6. Compound 20 was treated with sodium azide to give azide 22, which was then reduced to 4-aminobutyronitrile (24). The preparation of 6 from 24 was similar to that of 5 from 17. Compound 7 was synthesized in a similar manner (Scheme 6).

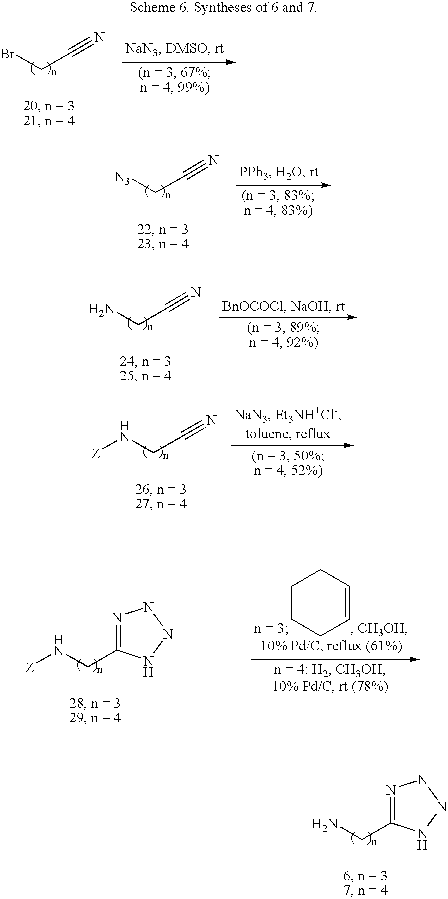

The substrate kinetic constants Kₘ and k_{cat} for the three tetrazoles (5-7) were determined by Hanes and Woolf plots, as known in the literature. (Woolf, B., cited by Haldane, J. B. S.; Stern, K. G. Algemeine Chemie der Enzyme; Steinkopf: Dresden, 1932; pp 119-120; Hanes, C. S. Biochem. J. 1932, 26, 1406-1421.) Compound 6, containing three methylene groups, has the highest k_{cat}/Kₘ value, indicating that 6 is the most efficient GABA-AT substrate with the optimal carbon chain length (Table 2).

TABLE 2
Kinetic constants for substrates 5-7

| Compound | Kₘ (mM) | k_{cat} (min⁻¹) | k_{cat}/kₘ (mM⁻¹ min⁻¹) |
|---|---|---|---|
| GABA | 2.4 | 49 | 20.4 |
| 5 | 2.3 | 15.9 | 6.9 |
| 6 | 2.4 | 28.6 | 11.7 |
| 7 | 8.0 | 41.6 | 5.2 |

Based on the structure of 6, a time-dependent inhibitor of GABA-AT (8) was designed and synthesized. The synthesis of a-vinyl-1H-tetrazole-5-propanamine (8) from 4,4-diethoxybutanenitrile (30) is shown in Scheme 7. Deprotection of 30 gave the 4-oxobutanenitrile (31), which was treated with vinylmagnesium bromide to give 32. The hydroxyl group in 32 was then converted to the phthalimide-protected amino group. The reaction of nitrile 33 with sodium azide resulted in tetrazole 34. Deprotection of 34 with 6 N HCl gave the desired compound 8.

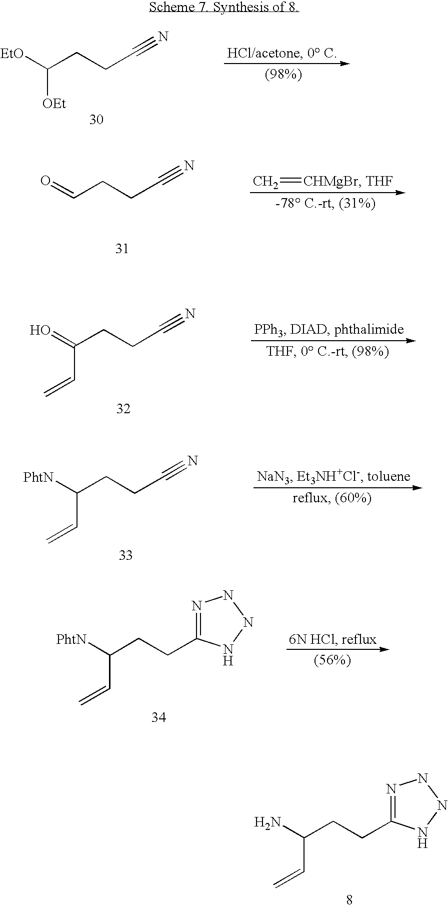

Referring to schemes 6 and 7, analogs of compound 8, where R₁ and R₂ can be independently selected from H, alkyl, and substituted alkyl, >can be prepared from the corresponding nitrile starting materials. Without limitation, R₁ and R₂ can be independently selected from C₁ to about C₄ alkyl and substituted (e.g., without limitation halogen, etc.) alkyl moieties. Such starting materials can be prepared, for instance, from the x-bromo-1-nitrile, with the appropriate reagent(s) to incorporate the R₁ and/or R₂ moieties, using synthetic techniques of the sort schematically illustrated above or straightforward modifications thereof known to those skilled in the art.

As expected, 8 showed time-dependent inhibition of GABA-AT, and its kinetic constants k_{inact} and K_{I} were determined by a Kitz and Wilson replot (Kitz, R.; Wilson, I. B. J. Biol. Chem. 1962, 237, 3245-3249.) to be 0.73 min⁻¹ and 5.6 mM, respectively (Table 3). The k_{inact} and K_{I} values of racemic vigabatrin were determined to be 2.2 min⁻¹ and 2.6 mM, respectively. Therefore, 8 is 6.6 times less efficient (k_{inact}/K_{I}) than vigabatrin.

TABLE 3
Kinetic constants for the time-dependent inhibitor 8 and vigabatrin

| Compound | K_{I} (mM) | k_{inact} (min⁻¹) | k_{inact}/k_{I} (mM⁻¹ min⁻¹) |
|---|---|---|---|
| vigabatrin | 2.6 | 2.2 | 0.86 |
| 8 | 5.6 | 0.73 | 0.13 |

The in vivo potency of enzyme inhibition, however, can strongly depend on the efficiency of the inhibitor to permeate the blood-brain barrier, which is related to the lipophilicity of the molecule. To determine an estimate of lipophilicity of these compounds, the log P values were calculated using Clog P software. The log P values calculated for 8 and vigabatrin are −0.47 and −2.217, respectively, which indicates that 8 has considerably higher lipophilicity and, therefore, higher potential permeability of the blood-brain barrier compared to vigabatrin.

It is possible that the tetrazole ring of 8 may exist either in a protonated or deprotonated form in the active site of GABA-AT, such that the deprotonated form can mimic a carboxylate anion. To determine the existence of the deprotonated form of the tetrazole ring in the enzyme active site methyl tetrazole derivatives 9 and 10, which cannot exist in a deprotonated form, were synthesized as shown in Scheme 8. The previously made compound 34 was treated with sodium hydride and iodomethane to give a mixture of 35 and 36, which were separated by column chromatography. Deprotection with 6 N HCl gave the desired compounds 9 and 10.

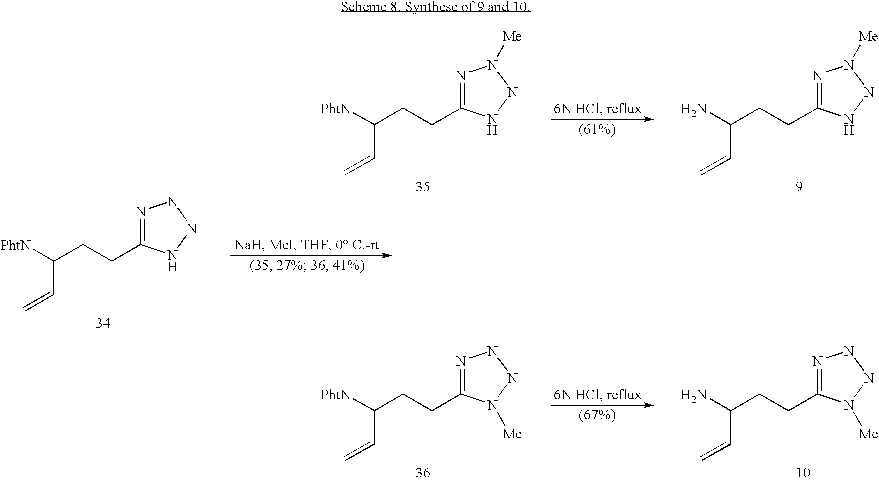

Neither 9 nor 10 showed time-dependent inhibition of GABA-AT in comparison with the corresponding tetrazole derivative 8 at the same concentration. Instead, both 9 and 10 were found to be weak time-independent inhibitors of GABA-AT with estimated IC₅₀ values for both greater than 10 mM. If the tetrazole ring of 8 were active in its protonated form in the active site of the enzyme, 9 and 10 would have inhibited the enzyme to a similar extent to that of 8. Without limitation to any one theory or mode of operation, the significantly decreased activities of 9 and 10 caused by methylation of the tetrazole, therefore, suggest that the tetrazole ring of 8 can exist in the deprotonated form in the enzyme active site.

### EXAMPLES OF THE INVENTION

The following non-limiting examples and data illustrate the various aspects and features relating to the compounds and/or methods of the present invention, including the preparation of various GABA-AT inhibitor compounds, as are available through the synthetic methodologies described herein. In comparison with the prior art, the present compounds and methods provide results and data which are surprising, unexpected and contrary thereto. While the utility of this invention is illustrated through the use of several compounds and molecular moieties incorporated therein, it will be understood by those skilled in the art that comparable results are obtainable with various other compounds and corresponding moieties, as are commensurate with the scope of this invention.

All chemicals, reagents and solvents were purchased from commercial sources (e.g., Sigma-Aldrich, Fisher Scientific, etc.) where available. Tetrahydrofuran was distilled over sodium metal under N₂, and dichloromethane was distilled over calcium hydride under N₂. Moisture sensitive reactions were carried out in oven-dried glassware, cooled under a N₂ atmosphere. Flash chromatography was performed with Merck silica gel 60 (230-400 mesh). Cation-exchange chromatography was performed on Dowex 50 resin (BioRad AG50W-X8, 100-200 mesh). ¹H and ¹³C NMR spectra were collected on Varian Mercury 400 MHz and Inova 500 MHz NMR spectrometers in the Analytical Service Laboratory at Northwestern University. High resolution mass spectra were obtained on a Finnigan MAT900XL mass spectrometer (EI) in the Analytical Services Laboratory at Northwestern University and on Micromass 70-VSE (EI) and Micromass Q-T of Ultima (ESI) mass spectrometers in the Mass Spectrometry Laboratory at the University of Illinois. Elemental analyses were obtained from Atlantic Microlab, Inc. (Norcross, Ga.). Enzyme assays were recorded on a Perkin-Elmer Lambda 10 UV-vis spectrophotometer. Radioactivity was measured by liquid scintillation counting using a Packard Tri-Carb 2100TR counter and Packard Ultima Gold XR scintillation cocktail.

### Example 1

### N-Cbz-β-alanyl chloride (12)

To a solution of N-Cbz-β-alanine (11, 1.0 g, 4.5 mmol) in dry methylene chloride (10 mL) was added 2.0 M oxalyl chloride solution in methylene chloride (20 mL, 40 mmol). The mixture was stirred at 25 ° C. for 4 h. Evaporation of the solvents gave 12 as a yellow oil (1.07 g, 99%). 1H NMR (400 MHz, CDCl₃), δ 7.4 (s, 5H), 5.1 (s, 2H), 3.5 (t, 2H, J=6.0 Hz), 3.2 (t, 2H, J=5.2 Hz).

### Example 2

### N-Cbz-methyl-β-alanylcarbamate (13)

Methyl carbamate (0.68 g, 9 mmol) was added to a solution of 12 (1.0 g, 4.4 mmol) in dry toluene (5 mL) at room temperature. The mixture was heated at 80 ° C. for 6 h, cooled, diluted with ethyl acetate (35 mL), and washed with water (2×30 mL) and brine (1×30 mL). The organic layer was dried with Na₂SO₄, filtered, and concentrated. The product was crystallized from ethyl acetate/hexanes to afford 13 as a white solid (0.46 g, 37%). ¹H NMR (400 MHz, CDCl₃), δ 7.3 (s, 5H), 5.1 (s, 2H), 3.8 (s, 3H), 3.5 (t, 2H, J=5.6 Hz), 3.0 (t, 2H, J=5.2 Hz). ¹³C NMR (100 MHz, CDCl₃), δ 73.6, 156.5, 152.2, 136.6, 128.7, 128.3, 128.2, 66.9, 53.4, 36.9, 36.1.

### Example 3

### Methyl β-alanylcarbamate (2)

A mixture of methyl N-Cbz-β-alanylcarbamate (13, 0.056 g, 0.2 mmol), 10% Pd/C (0.05 g), formic acid (88%, 0.15 mL), and methanol (7 mL) was stirred for 2 h at room temperature. The catalyst was removed by filtration through a Celite bed. The filtrate was concentrated to give the formate salt of 2 as a white solid (0.030 g, 78%). ¹H NMR (400 MHz, D₂O), δ 8.4 (s, 1H), 3.8 (s, 3H), 3.2 (t, 2H, J=6.0 Hz), 3.0 (t, 2H, J=5.6 Hz). ¹³C NMR (100 MHz, D₂O), δ 172.7, 169.2, 153.4, 53.3, 34.7, 33.1. HRMS (ESI): calculated for C₅H₁₁N₂O₃ (M+H)⁺: 147.0770. Found: 147.0773.

### Example 4

### Methyl N-Cbz-b-alanylsulfonamide (15)

A solution of 11 (2.23 g, 10 mmol) in dry THF (20 mL) was added dropwise to a stirred solution of carbonyldiimidazole (1.62 g, 10 mmol) in dry THF (20 mL) under N₂. The mixture was stirred for 30 min, refluxed for 30 min, and allowed to cool to room temperature. Methyl sulfonamide (0.95 g, 10 mmol) was added in one portion, and the mixture was stirred for 10 min before a solution of DBU (1.52 g, 10 mmol) in dry THF (10 mL) was added dropwise. The resulting mixture was stirred overnight and poured into ice-cold 1 N HCl (200 mL). The formed precipitate was filtered, washed with water, and dried to give 15 as a white solid (2.2 g, 73%). ¹H NMR (400 MHz, CDCl₃), δ 7.4 (s, 5H), 5.1 (s, 2H), 3.5 (tetra, 2H, J=6.0 Hz), 3.3 (s, 3H), 2.6 (t, 2H, J=4.8 Hz).

### Example 5

### Methyl b-alanylsulfonamide (3)

To 15 (1.0 g, 3.3 mmol) was added a solution of hydrogen bromide in acetic acid (30%, 10 g) with stirring. After 20 min, the mixture was slowly diluted to 100 mL with diethyl ether, and the liquids were decanted. The solid was resuspended in ether (100 mL) and stirred, and the suspension filtered and washed with ether to give 3 as a white solid (0.54 g, 71%). ¹H NMR (400 MHz, D₂O), δ 3.33 (s, 3H), 3.29 (t, 2H, J=6.0 Hz), 2.85 (t, 2H, J=6.4 Hz). ¹³C NMR (100 MHz, D₂O), δ 171.8, 40.8, 34.5, 32.5. HRMS (ESI): calculated for C₄H₁₁N₂O₃S (M+H)⁺: 167.0490. Found: 167.0497.

### Example 6

### Indole-5-methanamine (4)

To an ice-cold 1.0 M solution of LiAlH₄ in THF (18 mL, 0.018 mol) was added dropwise under N₂ a solution of 5-cyanoindole (16, 1.56 g, 0.011 mol) in dry THF (25 mL). After the addition was complete, the mixture was allowed to warm to room temperature and was stirred overnight. The resulting mixture was cooled in an ice bath, and excess LiAlH₄ was quenched with 10% NaOH. The product was extracted with ethyl acetate and dried over anhydrous magnesium sulfate. The solvent was removed by rotary evaporation to give the crude product (1.1 g), which was recrystallized from ethyl acetate/hexanes to give crystalline 5 (0.7 g, 45%). ¹H NMR (400 MHz, DMSO-d₆), δ 11.0 (s, 1H), 7.5 (s, 1H), 7.3 (d, 2H, J=8.4 Hz), 7.0 (d, 1H, J=8.4 Hz), 6.4 (s, 1H), 3.8 (s, 2H), 2-3 (br, 2H). HRMS (EI): calculated for C₉H₁₀N₂ (M⁺): 146.0838. Found: 146.0835.

### Example 7

### N-Cbz-3-aminopropionitrile (18)

3-Aminopropionitrile 17 (0.56 g, 8.0 mmol) was suspended in water (10 mL) and THF (10 mL). The pH was adjusted to 9.0 by addition of NaOH (0.2 g, 5 mmol). Benzyl chloroformate (1.7 g, 10 mmol) was added dropwise over 2 h at 20-25 ° C. to the resulting clear solution, and the pH was kept constant at 9.0 by addition of aqueous NaOH (4 M, 2.5 mL). The mixture was stirred for 1 h at pH 9.0, extracted with ethyl acetate, and dried with Na2SO4. The solvents were removed by rotary evaporation to give crude 18 (1.6 g, 98%) as an oil. ¹H NMR (400 MHz, DMSO-d₆), δ 7.3 (s, 5H), 5.0 (s, 2H), 3.22˜3.27 (m, 2H), 2.6 (t, 2H, J=6.4 Hz).

### Example 8

### N-Cbz-1H-tetrazole-5-ethanamine (19)

The mixture of N-Cbz-3-aminopropionitrile 18 (0.26 g, 1.3 mmol), triethylamine hydrochloride (0.38 g, 4 mmol), and sodium azide (0.26 g, 4 mmol) in toluene (10 mL) was heated to 95-100 ° C. for 24 h. After cooling, the product was extracted with water (20 mL). The separated aqueous layer was acidified with 1 N HCl to pH 1.5 to precipitate the produced tetrazole. The formed precipitate was filtered, washed with 1 N HCl, and dried under reduced pressure to give 19 (0.17 g, 56%) as a white solid. ¹H NMR (400 MHz, CD₃OD), δ 7.3 (s, 5H), 5.0 (s, 2H), 3.5 (t, 2H, J=4.8 Hz), 3.1 (d, 2H, J=5.6 Hz).

### Example 9

### 1H-Tetrazole-5-ethanamine (5)

A mixture of 19 (0.17 g, 0.7 mmol), 10% Pd/C (0.10 g), cyclohexene (4 mL), and methanol (6 mL) was refluxed overnight. The catalyst was removed by filtration through a Celite bed. The solvent was removed by rotary evaporation to give crude 5 as a white solid, which was purified by cation-exchange chromatography (AG® 50W-X8, eluting with 0.15 N HCl) to give pure 5 in the form of a hydrochloride salt (0.05 g, 63%). ¹H NMR (400 MHz, D2O), δ 3.3 (t, 2H, J=6.0 Hz), 3.1 (t, 2H, J=6.0 Hz). ¹³C NMR (100 MHz, D₂O), δ 159.0, 38.3, 22.7. Anal. Calcd for C₃H₈ClN₅ 0.2H₂O: C, 23.52; H, 5.53; N, 45.73. Found: C, 23.93; H, 5.42; N, 45.52.

### Example 10

### 4-Azidobutanenitrile (22)

Sodium azide (1.2 g, 18.5 mmol) was added to a solution of 4-bromobutyronitrile (20, 1.8 g, 12.0 mmol) in DMSO (20 mL). After 18 h of stirring at room temperature, water (40 mL) was added, and the solution was extracted with diethyl ether. Evaporation of the solvent gave 22 as a light yellow oil (0.9 g, 67%). 1H NMR (400 MHz, CDCl₃), δ 3.5 (t, 2H, J=6.0 Hz), 2.5 (t, 2H, J=7.2 Hz), 1.90˜1.95 (m, 2H).

### Example 11

### 4-Aminobutanenitrile (24)

Triphenylphosphine (2.11 g, 8.0 mmol) and water (0.2 mL) were added to 22 (0.9 g, 8.0 mmol) dissolved in THF (10 mL). After 18 h at room temperature, the solvent was removed by rotary evaporation. Ethyl acetate (30 mL) was added to the crude product, and the desired compound was extracted with 1 N HCl (30 mL). The aqueous phase was basified to pH 12 with 10% NaOH and was extracted with ethyl acetate (2×·30 mL). Evaporation of the solvent gave 24 as an oil (0.56 g, 83%). ¹H NMR (400 MHz, CDCl₃), δ 2.9 (t, 2H, J=6.8 Hz), 2.4, (t, 2H, J=7.2 Hz), 1.75-1.82 (m, 2H).

### Example 12

### 1H-Tetrazole-5-propanamine (6)

The synthetic procedure from 24 to 6 (4 mmol scale, 30% for three steps) is similar to that from 17 to 5. ¹H NMR (400 MHz, D₂O): δ 2.91-2.99 (m, 4H), 2.02-2.10 (m, 2H). ¹³C NMR (100 MHz, D₂O), δ 161.9, 38.9, 25.9, 21.5. Anal. Calcd for C₄H₁₀ClN_{5.}0.4H₂O: C, 28.13; H, 6.37; N, 41.00. Found: C, 28.53; H, 6.02; N, 40.81.

### Example 13

### 1H-Tetrazole-5-butanamine (7)

The synthetic procedure from 21 to 7 (12 mmol scale, 31% for five steps) is similar to that from 20 to 6. ¹H NMR (500 MHz, D₂O), δ 2.93-2.98 (m, 2H), 2.84-2.88 (m, 2H), 1.72-1.78 (m, 2H), 1.57-1.62 (m, 2H). ¹³C NMR (126 MHz, D₂O), δ 163.1, 39.3, 26.2, 24.9, 23.6. Anal. Calcd for C₅H₁₂ClN₅.0.5H2O: C, 32.18; H, 7.02; N, 37.52. Found: C, 32.39; H, 6.76; N, 37.29.

### Example 14

### 4-Oxobutanenitrile (31)

A mixture of 4,4-diethoxybutanenitrile (30, 0.95 g, 6.0 mmol), acetone (30 mL), and 6 N HCl (12 mL) was stirred at 0° C. for 9 h. After the reaction was complete, the mixture was concentrated to approximately 2 mL and was extracted with chloroform (4×·10 mL). The combined organic phase was dried with sodium sulfate. The solvent was removed by rotary evaporation to give crude 31 as an oil (0.49 g, 98%). ¹H NMR (400 MHz, CDCl₃), δ 9.8 (s, 1H), 2.9 (t, 2H, J=7.2 Hz), 2.6 (t, 2H, J=7.2 Hz).

### Example 15

### 4-Hydroxy-5-hexenenitrile (32)

A 1.0 M solution of vinylmagnesium bromide (5.9 mL, 5.9 mmol) in THF was added dropwise to a solution of crude 31 (0.49 g, 5.9 mmol) in dry THF (10 mL) at −78 ° C. The mixture was stirred at −78 ° C. for 1 h and was further stirred at room temperature overnight. Saturated aqueous NH₄Cl (15 mL) was added with stirring to the turbid solution chilled in an ice bath. The aqueous phase was extracted with ethyl acetate (3×15 mL), and the combined organic extracts were washed with water (10 mL) and brine (2×10 mL), dried with sodium sulfate, and concentrated under vacuum to give crude 32 as a yellow oil. The crude product was purified by chromatography on silica gel (ethyl acetate/hexanes, 4:6) to give a colorless oil (0.20 g, 31%). ¹H NMR (400 MHz, CDCl₃), δ 5.82-5.90 (m, 1H), 5.3 (d, 1H, J=17.6 Hz), 5.2 (d, 1H, J=10.0 Hz), 2.46-2.57 (m, 2H), 1.78-1.95 (m, 2H). ¹³C NMR (100 MHz, CDCl3), δ 139.5, 116.5, 71.3, 32.3, 13.6.

### Example 16

### 4-Phthalimido-5-hexenenitrile (33)

A solution of 32 (0.35 g, 3.1 mmol), triphenylphosphine (0.87 g, 3.3 mmol), and phthalimide (0.50 g, 3.3 mmol) in dry THF (15 mL) was stirred at 0° C. under N₂ for 10 min. A solution of diisopropyl azodicarboxylate (DIAD, 0.66 g, 3.3 mmol) in THF (8 mL) was added dropwise over 20 min. The mixture was stirred at room temperature for 3 h. After the solvent was removed by rotary evaporation, the crude product was purified by chromatography on silica gel (ethyl acetate/hexanes, 1:9) to give a mixture of 33 and diisopropyl hydrazodicarboxylate, a by-product formed from DIAD (1.19 g, ˜3:2 m/m, 97%). ¹H NMR (400 MHz, CDCl₃), δ 7.72-7.85 (m, 4H), 6.14-6.23 (m, 1H), 5.34 (d, 1H, J=17.2 Hz), 5.26 (d, 1H, J=10.4 Hz), 4.8 (tetra, 1H, J=5.6 Hz), 2.27-2.48 (m, 4H).

### Example 17

### 5-(3-Phthalimido-4-pantenyl)-1H-tetrazole (34)

The mixture of 33 and diisopropyl hydrazodicarboxylate prepared above (0.88 g, 2.6 mmol) was added to a solution of triethylamine hydrochloride (0.76 g, 8 mmol) and sodium azide (0.52 g, 8 mmol) in toluene (15 mL). After 18 h of stirring at 95-100_C, the cooled product was extracted with water (20 mL). The separated aqueous layer was acidified with 10% HCl to pH 1.5 to salt out the produced tetrazole. The formed precipitate was filtered and dried to give 34 as a light brown solid (0.44 g, 60%). ¹H NMR (400 MHz, CDCl₃), δ 7.8 (dd, 4H, J=35.2 Hz, 2.4 Hz), 6.22-6.29 (m, 1H), 5.27 (d, 1H, J=4.4 Hz), 5.24 (d, 1H, J=3.2 Hz), 4.72 (s, 1H), 3.19-3.22 (m, 1H), 2.78-2.84 (m, 1H), 2.62-2.71 (m, 1H), 2.18-2.22 (m, 1H).

### Example 18

### 1H-Tetrzole-5-(α-vinyl-propanamine) (8)

To a solution of 6 N HCl (20 mL) was added 34 (0.2 g, 1 mmol), and the mixture was refluxed for 6 h. The mixture was washed with ethyl acetate (2×20 mL). Evaporation of the solvent gave crude 8 as a yellow oil. To remove the trace amount of phthalic acid, the crude product was purified by cation-exchange chromatography (AG® 50W-X8, eluting with 0.2 N HCl) to give 8 in the form of a hydrochloride as a colorless oil. The hydrochloride was loaded on a second cation-exchange column, eluted with water followed by 0.15 N ammonium hydroxide to give the free amine form of 8 as a white solid (0.086 g, 56%). ¹H NMR (400 MHz, D₂O), δ 5.72-5.81 (m, 1H), 5.38-5.45 (m, 2H), 3.80 (br s, 1H), 3.00-3.07 (m, 2H), 2.24-2.32 (m, 1H), 2.09-2.19 (m, 1H). ¹³C NMR (400 MHz, D₂O), δ 155.1, 131.5,122.3, 53.2, 29.1, 19.0. Anal. Calcd for C₆H₁₁N₅.0.4H2O: C, 44.93; H, 7.42; N, 43.66. Found: C, 44.93; H, 7.38; N, 43.56.

### Example 19

### 2-Methyl-2H-tetrazole-5-(α-vinyl-N-phthaloylpropanamine) (35) and 1-methyl-1H-tetrazole-5-(α-vinyl-N-phthaloylpropanamine) (36)

A solution of 34 (0.167 g, 0.6 mmol) in dry THF (10 mL) was cooled to 0° C. NaH (60% in mineral oil, 0.034 g, 0.75 mmol) dissolved in dry THF (5 mL) was added dropwise over 20 min. The mixture was stirred for an additional 10 min, and iodomethane (0.11 g, 0.75 mmol) was added. After the mixture was stirred at room temperature for 2 h, H₂O (20 mL) was added, and the resulting mixture was extracted with EtOAc (2×·20 mL). The combined organic phases were washed with H2O (2×20 mL) and brine (2×20 mL), dried with Na₂SO₄, and the solvents were removed by rotary evaporation. The resulting mixture of 35 and 36 was separated by column chromatography on silica gel (EtOAc/hexanes, 4:6). Evaporation of the faster eluting fractions gave 35 as an oil (0.047 g, 27%). ¹H NMR (400 MHz, CDCl₃), δ 7.73-7.85 (m, 4H), 6.22-6.28 (m, 1H), 5.30 (d, 1H, J=17.2 Hz), 5.23 (d, 1H, J=10.4 Hz), 4.82 (tetra, 1H, J=8.0 Hz), 4.25 (s, 3H), 2.91 (tetra, 2H, J=8.0 Hz), 2.56-2.61 (m, 1H), 2.45-2.55 (m, 1H). Evaporation of the slower eluting fractions gave 36 as an oil (0.071 g, 41%). ¹H NMR (400 MHz, CDCl₃), δ 7.74-7.86 (m, 4H), 6.21-6.30 (m, 1H), 5.32 (d, 1H, J=17.6 Hz), 5.27 (d, 1H, J=10.0 Hz), 4.86 (tetra, 1H, J=5.6 Hz), 3.97 (s, 3H), 2.84-2.90 (m, 2H), 2.66-2.74 (m, 1H), 2.51-2.55 (m, 1H).

### Example 20

### 2-Methyl-2H-tetrazole-5-(α-vinyl-propanamine) (9) and 1-methyl-1H-tetrazole-5-(α-vinyl-propanamine) (10)

The deprotection procedures from 35 and 36 to 9 and 10, respectively, are similar to that from 34 to 8. Compound 9 (0.016 g, 61%). ¹H NMR (400 MHz, D₂O), δ 5.74˜5.81 (m, 1H), 5.35-5.40 (m, 2H), 4.25 (s, 3H), 3.76 (s, 1H), 2.87-2.91 (m, 2H), 2.14-2.22 (m, 1H), 2.02˜2.10 (m, 1H). ¹³C NMR (400 MHz, D2O), δ 165.1, 132.2, 122.1, 53.5, 33.6, 30.1, 20.9. HRMS (EI): calculated for C₇H₁₃N₅ (M⁺): 167.1171. Found: 167.1160. Compound 10 (0.027 g, 67%). ¹H NMR (400 MHz, D2O), δ 5.63-5.71 (m, 1H), 5.29-5.33 (m, 2H), 3.84 (s, 3H), 3.76-3.79 (m, 1H), 2.80-2.84 (m, 2H), 2.13-2.17 (m, 1H), 2.00-2.05 (m, 1H). ¹³C NMR (100 MHz, D2O) δ 155.2, 131.9, 122.5, 53.4, 33.6, 28.6, 19.0. HRMS (EI): calculated for C₇H₁₃N₅ (M⁺): 167.1171. Found: 167.1158.

### Example 21

### Enzyme and Assays

GABA-AT (1.88 mg/mL, specific activity 2.73 unit/mg) was purified from pig brain by the procedure described in the literature. (Churchich, J. E.; Moses, U. J. Biol. Chem. 1981, 256, 101-1104. (Succinic semialdehyde dehydrogenase (SSDH) was purified from GABAse, a commercially available mixture of SSDH and GABA-AT, using the procedure of Jeffery et al. (Jeffery, D.; Weitzman, P. D. J.; Lunt, G. G. Insect Biochem. 1988, 28, 347-349.) GABA-AT activity was assayed using a published method. (Scott, E. M.; Jakoby, W. B. J. Biol. Chem. 1958, 234, 932-936.) As the method was modified, the final assay solution consists of 11 mM GABA, 1.1 mM NADP⁺, 5.3 mM α-KG, 2 mM β-mercaptoethanol, and excess SSDH in 50 mM potassium pyrophosphate buffer, pH 8.5. The change in UV absorbance at the wavelength of 340 nm caused by the formation of NADPH is proportional to the GABA-AT activity.

### Example 22

### Substrate Activities of 2-7

Potential substrates 2-7 of varying concentrations (1-5 mM) were incubated with GABA-AT (17.1 μM, 5-7 lL) at 25° C. in 50 mM potassium pyrophosphate buffer, pH 8.5, containing 2 mM β-mercaptoethanol and 2.9 mM [5-¹⁴C]2-ketoglutarate (0.1 mCi/mmol) in a total volume of 100 μL. After incubation (48 h for the preliminary test, 1 h for determination of kinetic constants), the mixture was quenched with trichloroacetic acid. The resulting [¹⁴C]glutamate was isolated by cation-exchange chromatography, and the DPM (disintegration per minute) value was measured. Controls consisted of the entire incubation mixture with substrates omitted. The substrate kinetic constants k_{cat} and Kₘ were determined by the method of Hanes and Woolf, as referenced above.

### Example 23

### Time-Dependent Inhibition of GABA-AT by 8

GABA-AT (17.1 μM, 25 μL) was incubated with 8 (120 μL final volume, 1-2 mM) at 25 ° C. in 50 mM potassium pyrophosphate buffer solution, pH 8.5, containing 2 mM α-ketoglutarate and 2 mM β-mercaptoethanol. Aliquots (20 μL) were withdrawn at timed intervals and were added immediately to the assay solution (575 μL) followed by the addition of excess SSDH (5 μL). The reaction rates were measured by a UV-vis spectrophotometer at 340 nm. Racemic vigabatrin was tested under the same conditions. A Kitz and Wilson replot was used to determine the kinetic constants k_{inact} and K_{I}, as referenced above.

### Example 24

### Time-Independent Inhibition of GABA-AT by 9 and 10

GABA-AT (17.1 μM, 5 μL) was assayed for its activity at 25° C. with varying concentrations (1-10 mM) of 9 and 10. The percentage of remained enzyme activity was obtained by comparison to that of an untreated enzyme control. The logarithm of the percentage of remained activity is plotted versus the concentration of the inhibitors to calculate IC₅₀ values.

As illustrated, above, representative substrates of GABA-AT containing bioisosteres of the carboxylic acid group with higher lipophilicity were synthesized and tested, with the tetrazole derivative shown to be most efficient. The tetrazole group, therefore, was selected as the carboxylate bioisostere for incorporation into a time-dependent GABA-AT inhibitor. Without limitation, the optimal carbon chain length of the tetrazole derivatives was determined. Representative compound 8, a more lipophilic analogue of the antiepilepsy drug vigabatrin, showed time-dependent inhibition of GABA-AT. Such compounds have a potency similar to that of vigabatrin (about one-sixth the potency), are more lipophilic (Clog P value) and are potentially more easily able to cross the blood-brain barrier.

We claim:

1. A γ-aminobutyric acid aminotransferase inhibitor compound selected from compounds of a formula 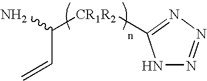
wherein n is an integer ranging from 1 to about 6; and R₁ and R₂ are independently selected from H, alkyl and substituted alkyl moieties, and tautomers and salts thereof.

2. The inhibitor compound of claim 1 wherein n is selected from 1, 2 and 3.

3. The inhibitor compound of claim 1 selected from R and S stereochemical configurations.

4. The inhibitor compound of claim 1 selected from ammonium salts of said compound.

5. The inhibitor compound of claim 1 wherein said compound is an ammonium salt, and the counter ion is the conjugate base of a protic acid.

6. The inhibitor compound of claim 1 wherein said tetrazole moiety is alkylated.

7. The inhibitor compound of claim 6 selected from ammonium salts of said compound.

8. An enzyme-inactivator complex comprising the addition product of a γ-aminobutyric acid aminotransferase and a compound selected from compounds of a formula 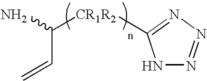
wherein n is an integer ranging from 1 to about 6; and R₁ and R₂ are independently selected from H, alkyl and substituted alkyl moieties, and tautomers and salts thereof.

9. The enzyme-inactivator complex of claim 8 wherein n is selected from 1, 2 and 3.

10. The enzyme-inactivator complex of claim 8 wherein said compound is selected from R and S stereochemical configurations.

11. The enzyme-inactivator complex of claim 8 wherein said tetrazole moiety is deprotonated.

12. The enzyme-inactivator complex of the claim 8 wherein said addition product further comprises a pyridoxal-5′-phosphate cofactor.

13. A method of inhibiting a γ-aminobutyric acid aminotransferase comprising contacting a γ-aminobutyric acid aminotransferase with an effective amount of at least one compound selected from compounds of a formula 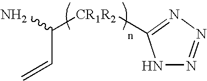
wherein n is an integer ranging from 1 to about 6; and R₁and R₂ are independently selected from H, alkyl and substituted alkyl moieties, and tautomers and salts thereof.

14. The method of claim 13 wherein n is selected from 1, 2 and 3; and R₁ and R₂ are H.

15. The method of claim 13 wherein compound is selected from R and S stereochemical configurations.

16. The method of claim 13 wherein said aminotransferase enzyme comprises a pyridoxal-5′-phosphate cofactor.

17. The method of claim 13 wherein said compound is selected from ammonium salts of said compound.

18. The method of claim 13 wherein said compound is present in an amount at least partially sufficient to provide time-dependent inhibition of said aminotransferase.

19. A method of using a tetrazole moiety to enhance the lipophilicity of a γ-aminobutyric acid aminotransferase, said method comprising:
providing a compound selected from compounds of a formula 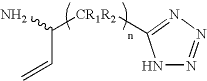
wherein n is an integer ranging from 1 to about 6; and R₁ and R₂ are independently selected from H, alkyl and substituted alkyl moieties, and tautomers and salts thereof; and
determining the lipophilicity of said compound, said lipophilicity compared to the lipophilicity of vigabatrin.

20. The method of claim 19 wherein n is selected from 1, 2 and 3.
